(19) United States
(12) Patent Application Publication
(10) Pub. No.: US 2026/0363916 A1
(43) Pub. Date: Nov. 19, 2026

(54) ADHESIVE BANDAGE SYSTEM FOR MEDICATION DELIVERY, TREATMENT-SITE IDENTIFICATION, AND TACTILE DISTRACTION

(71) Applicant: Stephen Welsh, Hurricane, UT (US)

(72) Inventor: Stephen Welsh, Hurricane, UT (US)

(21) Appl. No.: 19/750,842

(22) Filed: Jul. 22, 2026

Related U.S. Application Data
(63) Continuation-in-part of application No. 18/065,214, filed on Dec. 13, 2022, now abandoned.
(60) Provisional application No. 63/288,852, filed on Dec. 13, 2021.

(51) Int. Cl. A61F 13/00; A61M 5/42; A61M 21/00; A61M 21/02

(52) U.S. Cl. CPC A61F 13/00063; A61F 13/00059; A61M 5/422; A61M 21/02; A61F 2013/00417; A61F 2013/00553; A61F 2013/00646; A61F 2013/00846; A61F 2013/00906; A61M 2021/0022; A61M 2021/0027

(57) ABSTRACT

An adhesive bandage system includes a first member configured to adhere to skin, a removable center region, a medication component, and a tactile distraction element positioned around a treatment window exposed by removal of the removable center region. The medication component may include a sealed reservoir configured to preserve a topical medication in an airtight or substantially airtight storage state and transition, upon activation, to an occlusive delivery state in which the medication contacts a treatment site. The bandage system may include a timing indicator and a skin-contact confirmation indicator. The tactile distraction element provides non-painful tactile stimulation adjacent to the treatment site when pressed. Removal of the removable center region exposes the conditioned treatment site for cleansing, visualization, targeting, and access during a needle stick, intravenous placement, or other medical procedure.

### CROSS-REFERENCE TO RELATED APPLICATIONS

This application is a continuation-in-part of U.S. Non-Provisional App. Ser. No. 18/065,214, filed on Dec. 13, 2022, which claimed the benefit of U.S. Provisional Application Ser. No. 63/288,852, filed on Dec. 13, 2021, both of which are incorporated herein by reference.

### TECHNICAL FIELD

The present disclosure relates to bandages. More particularly, the present disclosure relates to an adhesive bandage system having a removable member or portion for medication delivery and situs indication, and further relates to adhesive bandage systems configured to provide tactile distraction, site preparation, and access to a prepared treatment site for injections, intravenous access, or other medical procedures.

### BACKGROUND

Topical medications are used in a variety of settings and with a variety of patients. The most common type of topical medication is simply an ointment or cream that is applied directly to the skin. While this can be an effective delivery method, it is often messy, as the ointment or cream may come into contact with a patient's clothes or other surfaces. In some instances, medication may also be inadvertently transferred to someone else, which may have serious consequences. Often times, the ointment or cream can be mistakenly rubbed off, decreasing the effectiveness of the medication. Additionally, it is difficult to produce a metered dose when simply applying directly to the skin without coverings.

To solve this issue, some topical medications are delivered using transdermal patches. However, conventional patches may not provide a clear indication of the exact skin location that has received the medication, and may not cooperate with the later workflow of cleansing, visualizing, and accessing the same treated location for a needle stick or other medical procedure. Further, many patches are not configured to remain as a surrounding target structure after a medication-delivery portion is removed.

Additionally, it may be beneficial for some ointments, creams, or patches to be removed after a short time so that other medications may be administered or so that a needle may be inserted into a prepared area. For example, a topical anesthetic such as lidocaine may be useful for numbing an injection site prior to an injection. However, topical anesthetics typically must be applied some time in advance of the injection to be effective. If a patient applies a cream or ointment beforehand, the medication may be rubbed off before arriving. If applied by the medical practitioner, the patient may occupy clinical time and space while waiting for the topical medication to take effect. Further, even if the topical anesthetic is used, once it is cleared from the skin, it may be difficult to identify the precise numbed location, resulting in injections outside the numbed location.

Pain associated with needle sticks may also be influenced by tactile or sensory factors. Non-painful tactile stimulation applied near a procedure site may help distract a patient from the needle stick and may reduce perceived pain by providing competing sensory input. Existing distraction approaches, however, often require separate handheld devices, batteries, cleaning, storage, retrieval, or active positioning by a provider or patient. Separate tools may interrupt the clinical workflow, may be inconsistently positioned relative to the injection site, and may require additional hands or steps at the time of injection.

In addition, topical creams and ointments may be sensitive to storage and use conditions. Exposure to air, moisture, evaporation, or other environmental conditions may alter viscosity, drug distribution, drug stability, crystallization, permeation, or other delivery characteristics before or during use. Accordingly, there is a need for a bandage system that can store a cream, ointment, gel, solution, or other medication in a sealed reservoir before use and then transition, upon activation, to an occlusive delivery configuration that maintains the medication against the skin during a treatment period.

Accordingly, there is a need for an adhesive bandage system that can deliver or maintain a topical medication at a selected treatment site, identify the treated location after removal of a center portion, facilitate skin preparation and workflow for a provider, and provide an integrated tactile distraction element surrounding the injection site. There is also a need for such a system that can provide combination therapy or sensory stacking, including local anesthesia and non-painful tactile stimulation, without requiring a separate distraction device. The present disclosure seeks to solve these and other problems.

### SUMMARY OF EXAMPLE EMBODIMENTS

In some embodiments, an adhesive bandage system comprises a first member and a second member, the second member removably replaceable within an aperture of the first member. The first member comprises an adhesive on a first side for adhering to the skin of a patient with an aperture in the center thereof. In some embodiments, the second member comprises an adhesive along its outer perimeter on a first side, the outer perimeter sized so as to overlap with the first member.

In some methods of use, a user may adhere the first member to a patient. The user may then place the desired medication in the aperture in an amount and dosage appropriate to the patient and treatment, the medication being in direct contact with the skin. The second member may then be placed so as to cover the aperture, the edges of the second member contacting the first member to removably adhere the second member to the first member. The second member may be removed to either add medication, replace the medication with a new medication, or to have access to an injection site.

In some embodiments, the second member may be preassembled to the first member. A user may then deposit the medication, on a bottom side, into the aperture that is covered by the second member. The first member may then be adhered to a patient. When desirable, a user may remove the second member to expose the skin. If additional medication is deposited, a second member may then be replaced on the first member.

In some embodiments, a medication may be sealed in the aperture by the manufacturer to form an airtight or substantially airtight medication reservoir. The reservoir may preserve a cream, ointment, gel, solution, topical anesthetic, lidocaine-containing formulation, or other medication before use by reducing exposure to air, moisture, evaporation, contamination, crystallization, or degradation. A user may then remove a release liner, rupture or open a seal, peel a cover, fold or press an activation region, or otherwise activate the bandage to transition the medication reservoir from a sealed storage state to a delivery state in which the medication contacts the skin. In the delivery state, the first member, second member, release liner, cover, reservoir wall, or other portion of the bandage may create an occlusive or substantially occlusive environment over the medication to maintain the medication at the selected treatment site during a treatment period.

In some embodiments, rather than placing an ointment or cream within the aperture, the second member may be a patch comprising medication.

In some embodiments, the adhesive bandage system comprises a target bandage having a surrounding tactile distraction element and a removable center region. In some embodiments, the removable center region comprises or is formed by a lidocaine patch or other topical anesthetic patch that conditions a treatment area. In other embodiments, the removable center region may support, cover, carry, or cooperate with a separate lidocaine patch or other topical anesthetic component. After a sufficient numbing period, the removable center region, including the lidocaine patch in some embodiments, may be removed to expose the conditioned treatment area while the surrounding tactile distraction element remains adhered to the patient around the injection site.

In some embodiments, the tactile distraction element surrounds the injection site in a target-ring architecture. The tactile distraction element may be configured so that pressure applied by a provider while stabilizing a patient's arm or other anatomical site activates the tactile distraction element and creates a plurality of non-painful tactile stimuli adjacent to the injection site. In this manner, the adhesive bandage system may provide combined pain-reduction mechanisms, including local anesthesia from the medication component and sensory distraction from the tactile distraction element.

In some embodiments, the adhesive bandage system comprises a radial perforation, slit, separable seam, separable annulus, or other separation feature extending through at least a portion of the first member and/or tactile distraction element. The separation feature may allow the surrounding bandage structure to be opened or separated after placement of an intravenous line, catheter, tube, needle, or other medical implement through the treatment window. Accordingly, the adhesive bandage system may be removed from the patient without disturbing the placed intravenous line, catheter, tube, needle, or other medical implement.

In some embodiments, the adhesive bandage system comprises one or more integrated indicators. For example, a timing indicator may provide a visual indication that a predetermined dwell time, anesthesia time, medication-delivery time, or treatment period has elapsed after the bandage is applied to the skin or after the medication reservoir is activated. In some embodiments, a skin-contact confirmation indicator may provide a visual indication that the medication component, adhesive, reservoir, or treatment region is adequately contacting the skin.

### BRIEF DESCRIPTION OF THE DRAWINGS

FIG. 1 illustrates a top perspective view of an adhesive bandage system;

FIG. 2 illustrates a bottom perspective view of an adhesive bandage system;

FIG. 3 illustrates a side perspective view of an adhesive bandage system;

FIG. 4 illustrates a top perspective view of an adhesive bandage system adhered to a patient;

FIG. 5 illustrates a second member of an adhesive bandage system being removed from a first member;

FIG. 6 illustrates a first member of an adhesive bandage system adhered to a patient with an exposed aperture;

FIG. 7 illustrates a bottom perspective view of an adhesive bandage system receiving medication in an aperture;

FIG. 8 illustrates a first member of an adhesive bandage system adhered to a patient with medication being deposited on the skin through an aperture;

FIG. 9 illustrates a rear, disassembled view of an adhesive bandage system;

FIG. 10 illustrates a side, front perspective view of an adhesive bandage system with a port;

FIG. 11 illustrates a rear, disassembled view of an adhesive bandage system comprising a transdermal patch;

FIG. 12 illustrates a side, disassembled view of an adhesive bandage system;

FIG. 13 illustrates a front, side perspective view of an adhesive bandage system on a user;

FIG. 14 illustrates a front, side perspective view of an adhesive bandage system on a user receiving an injection;

FIG. 15 illustrates a side, disassembled view of an adhesive bandage system; and

FIG. 16A illustrates a side, disassembled view of an adhesive bandage system;

FIG. 16B illustrates a side, detailed view of an adhesive bandage system;

FIG. 16C illustrates a side, detailed view of an adhesive bandage system;

FIG. 17 illustrates a top view of an adhesive bandage system having a target bandage with a tactile distraction element surrounding a removable center region;

FIG. 18 illustrates a side elevation view of an adhesive bandage system applied to a patient's skin;

FIG. 19 illustrates an adhesive bandage system in which a tactile distraction element comprises a textured or hook-like material positioned around a central target area, with a provider pressing on the tactile distraction element;

FIG. 20 is a detailed view of a tactile distraction structure interacting with the skin of a patient;

FIG. 21 illustrates an adhesive bandage system having one or more snap domes or clicker structures positioned around a removable center region and configured to provide tactile stimulation when pressed by a provider;

FIG. 22 illustrates an adhesive bandage system having micro-hooks or other protrusions positioned on a tactile distraction element around a removable center region;

FIG. 23 illustrates an adhesive bandage system having microbeads or other surface protrusions positioned on a tactile distraction element around a removable center region; and

FIG. 24 illustrates an adhesive bandage system having one or more tactile actuation structures positioned around an injection site, with a radial perforation for IV lines.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As discussed earlier, there is a need for an adhesive bandage system that can deliver or maintain topical medications, creams, ointments, solutions, patches, or reservoir-based formulations at a selected treatment site; preserve medication before use; provide an occlusive delivery environment during use; identify the treated location after medication delivery; confirm timing or skin contact; and assist with tactile distraction, cleansing, targeting, and access to a prepared treatment site. The bandage systems disclosed herein seek to solve these and other problems.

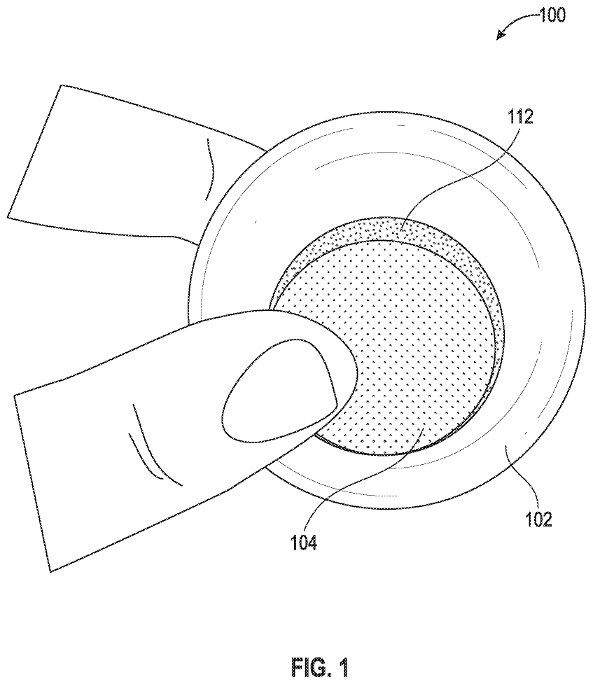

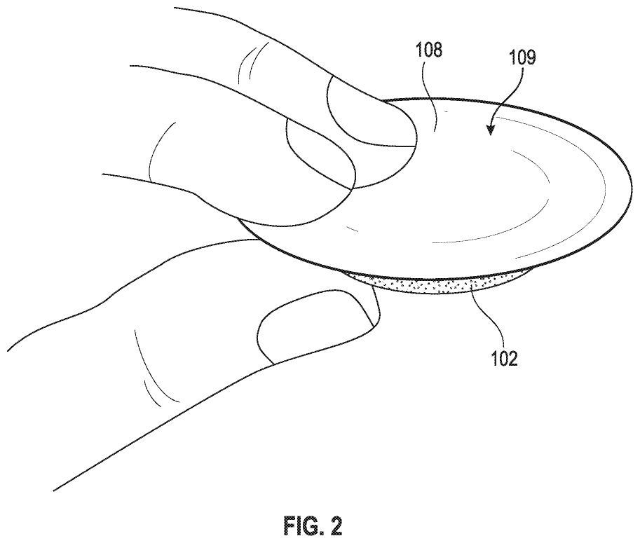

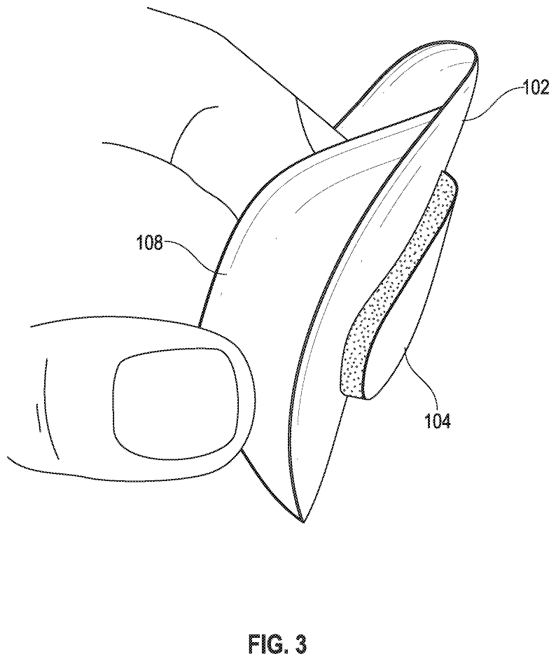

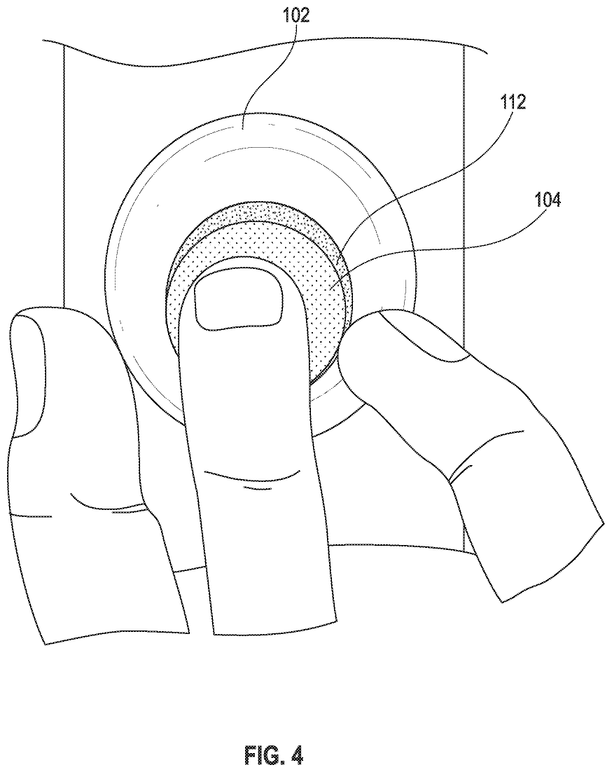

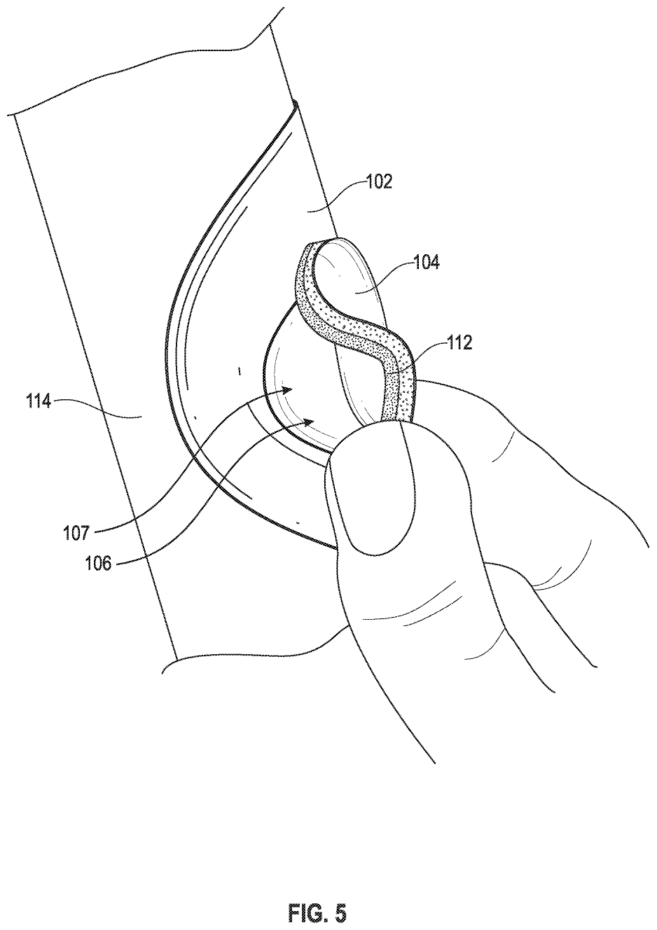

Referring to FIGS. 1-7, in some embodiments, an adhesive bandage system 100 comprises a first member 102, a second member 104, and an aperture 106 (FIG. 5) in the first member 102. The second member 104 is configured to cover and/or be at least partially receivable within the aperture 106. In some embodiments, the first member 102 comprises a release liner 108, such as a plastic or paper film, on a first side 109, that may form a protective layer over an adhesive on the first side 109 (underside) of the first member 102 for adhering to the skin of a patient. The release liner may be substantially coextensive with the adhesive layer. As shown in FIG. 2, the release liner 108 may cover the entire bottom of the first side 109, including the aperture 106.

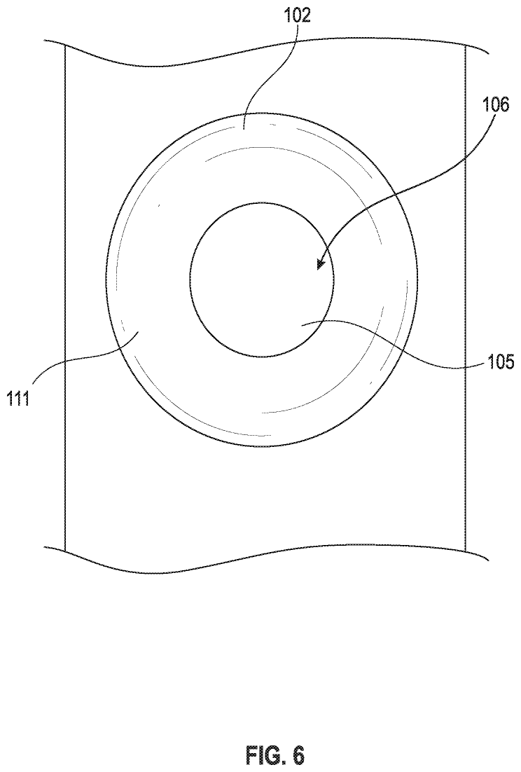

A medication (e.g., lidocaine) may be deposited in the aperture 106 in several ways for contacting the patient's skin. For example, as shown in FIGS. 1-6, in some embodiments, a manufacturer may place a desired medication or substance in the aperture 106 in advance, with the medication/substance remaining sealed between the release liner 108 and the second member 104 until use. In other words, a medication reservoir 107 (FIG. 5) is formed between the release liner 108 and the second member 104. To apply the adhesive bandage system 100 to a patient, a user peels the release liner 108 from the first side 109, thereby exposing an adhesive layer on the first side 109 and also exposing the aperture 106, thereby allowing the medication/substance in the medication reservoir 107 to contact a patient's skin 105. After a predetermined timeframe (e.g., sufficient to numb the skin), the user may remove, as shown in FIG. 5, the second member 104 from the first member 102, thereby exposing the aperture 106 from a top, second side 111 of the first member 102, as best seen in FIG. 6. The user may then clean the skin 105 through the aperture 106 in preparation for the injection or other procedure. Because the first member 102 remains adhered to the skin, the user is able to easily determine the area for the injection by injecting through the aperture 106 (the “bullseye”).

In some embodiments, the medication reservoir 107 is configured as an airtight or substantially airtight storage compartment. For example, the medication reservoir 107 may be bounded by the release liner 108, the second member 104, a backing layer, a membrane, a frangible seal, a peripheral heat seal, an adhesive seal, a foil layer, a polymer film, or combinations thereof. The sealed storage compartment may preserve the medication before use by reducing air exchange, moisture exchange, solvent evaporation, contamination, drying, crystallization, oxidation, or other changes that may reduce shelf life or drug-delivery performance. In some embodiments, the medication reservoir 107 itself forms the storage package for the medication, such that the medication is stored within the bandage system 100 rather than merely being stored in a separate external pouch.

In some embodiments, the adhesive bandage system 100 is activatable to transition the medication reservoir 107 from a sealed storage state to an occlusive delivery state. Activation may include peeling away the release liner 108, removing a barrier film, rupturing a frangible seal, breaking a blister, pressing a reservoir wall, opening a valve, separating two sealed layers, folding a portion of the bandage, or otherwise causing the medication to be exposed for contact with the skin 105. After activation and placement on the skin 105, the first member 102, second member 104, release liner 108, or other cover portion may cooperate to hold the medication against the skin 105 in an occlusive or substantially occlusive environment. This configuration may reduce evaporative loss during use and may maintain a desired viscosity, hydration, distribution, concentration, or drug-delivery condition of the medication during the treatment period.

In some embodiments, the medication reservoir 107 is peel-activated. For example, peeling the release liner 108, a cover, or another barrier layer from the adhesive bandage system 100 may simultaneously open, break, disrupt, or unseal an airtight or substantially airtight storage seal and expose or position the medication for contact with the skin 105. In this manner, a single peel-to-activate action may transition the medication reservoir 107 from the sealed storage state to the occlusive delivery state without requiring a separate step of rupturing a blister, pressing a reservoir wall, opening a valve, or separately breaking a frangible seal.

In some embodiments, the medication reservoir 107 contains a topical anesthetic cream, such as a lidocaine cream, a lidocaine-containing formulation, an anesthetic mixture, or another topical numbing composition. The reservoir 107 may be sized to contain a predetermined dose, volume, thickness, or surface area of the medication so that, when the bandage is applied, the medication contacts a defined treatment site corresponding to the aperture 106. In some embodiments, the reservoir 107 may be prefilled during manufacture; in other embodiments, the reservoir 107 may be filled by a clinician, caregiver, patient, or other user before application.

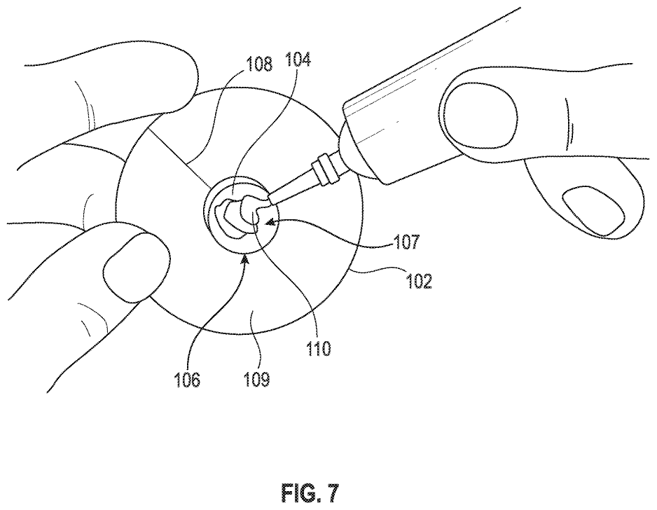

In some embodiments, rather than the manufacturer placing medication or substances in the medication reservoir 107, the medication reservoir 107 may be accessible to a user, as shown in FIG. 7, to add the desired medication 110, with the second member 104 adhered to the first member 102 to contain the medication 110 in the formed medicine reservoir 107. In other words, the release liner 108 in this embodiment does not cover the aperture 106, leaving it accessible to a user. Once the medication 110 is deposited, a user will remove the release liner 108 from the first side 109 and will adhere the first side 109 of the first member 102 to a patient. When a sufficient time has elapsed for the medication to be absorbed, a user may then remove the second member 104, thereby exposing the aperture 106 and the medicine 110 therein. A user may then clean out the medicine and administer the injection or perform a procedure through the aperture 106, which forms the “bullseye” for the user.

It will be appreciated that any suitable substances may be added to the aperture 106 or reservoir 107, such as antiseptics, antifungal agents, antibacterial agents, antiviral agents, hormones, antihistamines, steroids, or any other substance desirable for transdermal application.

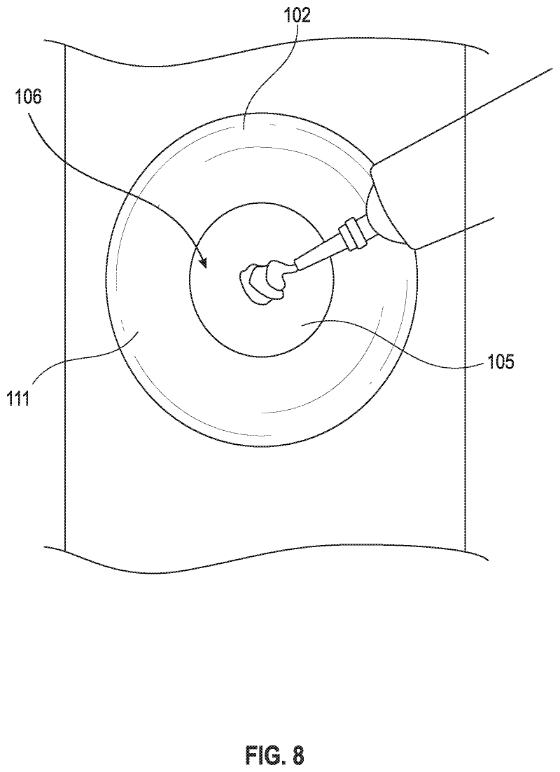

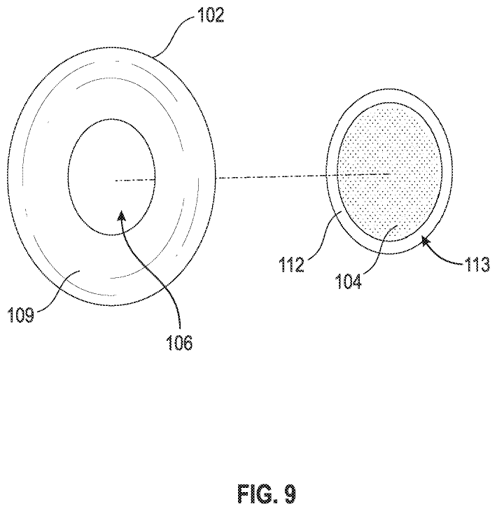

In some embodiments, as shown in FIGS. 8-9, a user may remove a release liner 108 from a first side 109 of the first member 102, adhere the first member 102 on a patient, deposit the medication 110 through the aperture 106 directly onto the skin 105, then seal the aperture 106 using the second member 104, thereby enclosing the medication 110 within the aperture 106 and on the skin 105 of a patient. Accordingly, in some embodiments, as shown in FIG. 9, the second member 104 comprises an adhesive along its outer perimeter 112 on a first side 113, the diameter of the outer perimeter 112 greater than the diameter of the aperture 106, thereby allowing the outer perimeter 112 to adhere to the second side 111 of the first member 102, thereby enclosing the medication 110 in the aperture 106, preventing the medication 110 from inadvertent or premature removal. The second member 104 may be removed to either add medication 110, replace the medication 110 with a new medication 110, or to have access to an injection site for inserting a needle or other medical procedure.

A user may remove the second member 104 to expose the aperture 106 and the skin 105 of the patient. Because the first member 102 remains adhered to the skin, a user or medical provider is able to easily determine where the medication has been in contact with the skin and, therefore, the injection site. For example, for an injection, an anesthetic may be placed in the medication reservoir 107 of the first member 102. Because the reservoir 107 remains covered by the second member 104, the medication is not rubbed off or otherwise prematurely removed from the desired site. When the patient is ready for the injection, the second member 104 may be removed (e.g., peeling it off from the first member 102), exposing the skin and residual medication 110. The aperture 106 allows the skin to be cleaned and sterilized. However, because the first member 102 remains adhered to the subject, the medical provider may confidently inject into the numbed and cleansed skin, the aperture 106 forming a target or bullseye for the injection. Unlike the prior art, which requires injections through layers of adhesive or other film, the adhesive bandage system 100 allows a medical provider to confidently inject directly into the prepared skin of the patient.

Additionally, after an injection, other medications 110 may be inserted into the aperture 106 if desired, such as topical analgesics and/or antibiotics. If additional medication 110 is deposited, a new, non-contaminated, second member 104 may then be replaced on the first member 102, once again sealing the aperture 106 and the medication therein, which forms the medicine reservoir 107.

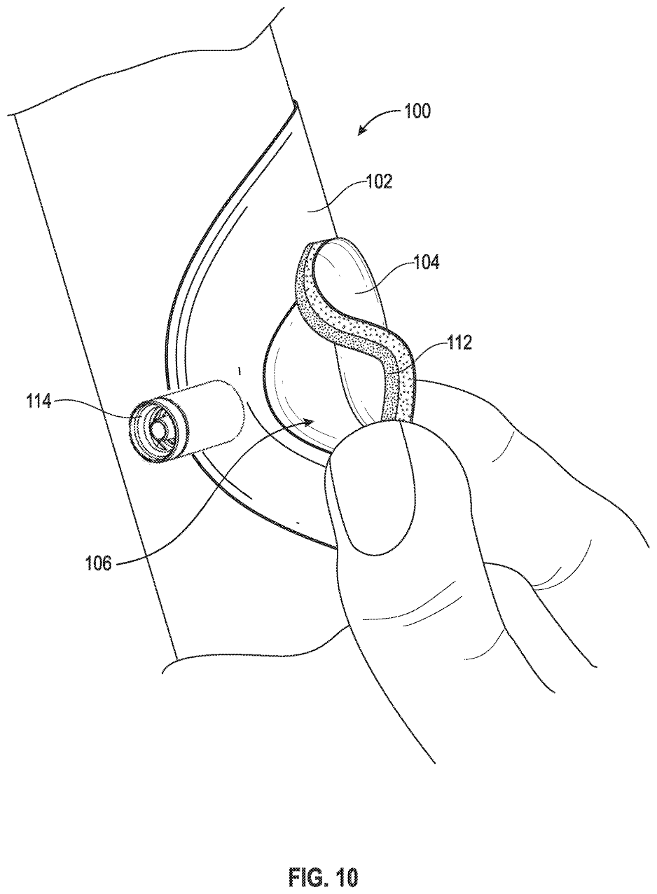

As shown in FIG. 10, in some embodiments, the adhesive bandage system 100 may comprise one or more ports or couplers 114 to connect to, or otherwise secure, other items, such as an IV catheter, tube, needle, etc., to the adhesive bandage system 100. The one or more couplers 114 may be positioned on the first member 102 so as to remain secured to a patient when the second member 104 is removed therefrom. In some embodiments, however, the second member 104 may be configured to receive tubes, needles, catheters, or other accessories as well. The one or more couplers 114 may be configured to anchor and secure catheters to prevent movement and dislodgment of the needle. The one or more couplers 114 may be secured by additional medical dressings and adhesives.

It will be appreciated that the size and shape of the adhesive bandage system 100 may vary for specific uses. Additionally, the first member 102 and/or the second member 104 of the adhesive bandage system 100 may comprise an occlusive or non-occlusive dressing, depending upon the application and use with different topical agents. The occlusive dressing may be an air- and water-tight trauma medical dressing made with a waxy coating as to provide a total seal around the patient's skin. The occlusive dressing may also comprise films, foams, hydrogels, or hydrocolloids. In some embodiments, the non-occlusive dressing may be gauze having natural or synthetic fibers such as cotton, rayon, or polyester. Any topical medication 110 may be used, including, but not limited to, lidocaine, benzocaine, testosterone, estrogen, estradiol, ketoprofen, indomethacin, diclofenac, capsaicin, methyl salicylate, topical antibiotics, gabapentin, or topical corticosteroids.

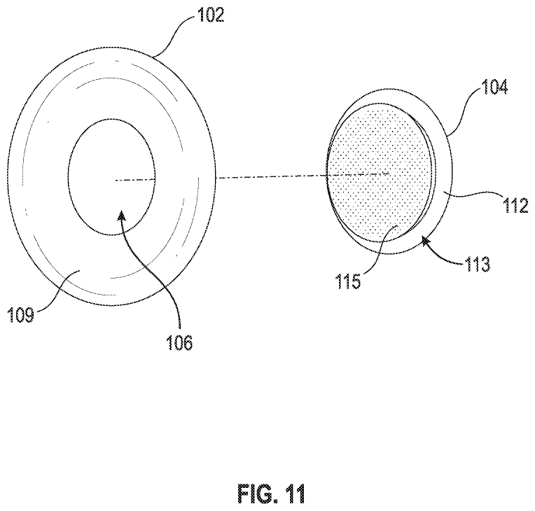

In some embodiments, as shown in FIG. 11, the second member 104 comprises a transdermal patch 115. In other words, rather than depositing medication into the aperture 106 and medicine reservoir 107, the transdermal patch 115 of the second member 104 is received through the aperture 106 where it contacts the skin of a patient. The outer perimeter 112 adheres to the second side of the first member 102, ensuring the transdermal patch 115 remains in contact with the skin. After a predetermined amount of time has elapsed, the second member 104 may be removed by peeling it from the first member 102, the skin may then be cleaned, and the procedure administered through the aperture 106, the aperture 106 functioning as a bullseye.

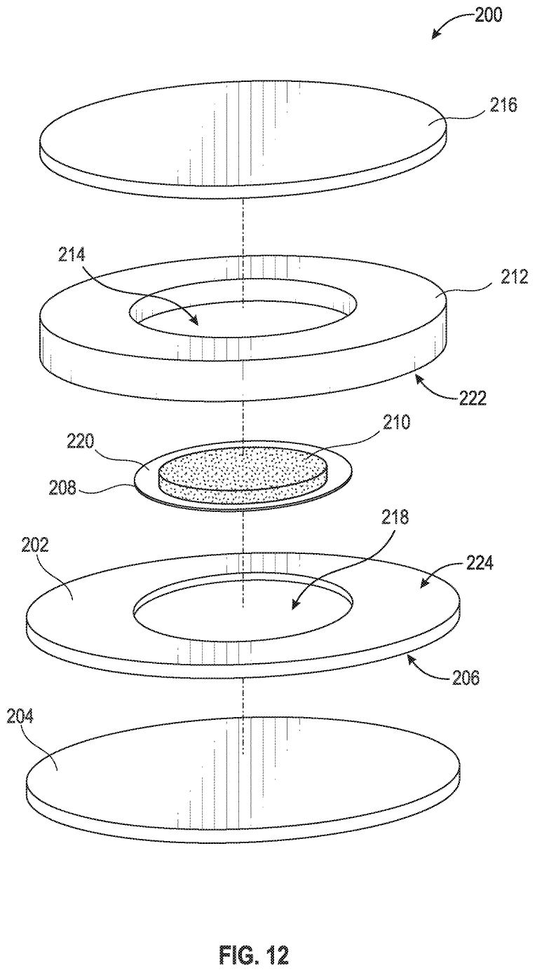

Referring to FIG. 12, in some embodiments, an adhesive bandage system 200 comprises a first member 202, a release liner 204 (e.g., backing paper) coupled to a first side 206 of the first member 202, and a second member 208 comprising medication or other topical ointment/cream 210. The second member 208 may comprise gauze or other non-stick material that allows for the medication to pass therethrough. A third member 212 may protect a pre-packaged medication 210. For example, the third member 212 may comprise an aperture 214 in the center thereof and have a height greater than the height of the medication 210 on the second member 208, thereby allowing the medication to reside within the aperture 214 during packaging and storage, reducing or preventing the medication 210 from coming into contact with a cover 216. The third member 212 may comprise foam or other materials, allowing it to flex, allowing for the bandage to be used on different curves of a patient's body. The third member 212 is manufactured from non-absorbent materials so that the medication is not absorbed thereinto. Lastly, a film or cover 216 encloses and protects the medication 210.

The second member 208 is received within a first aperture 218 of the first member 202. Because the second member 208 is made from gauze, mesh, or other material allowing the medication 210 to pass or seep therethrough, the medication 210 will contact a patient once the release liner 204 is removed. The second member 208 is adhered, around an outer edge 220, to the third member 212. The underside 222 of third member 212 is releasably adhered to top side 224 of the first member 202, allowing a user to remove the third member 212 by peeling it from the first member 202.

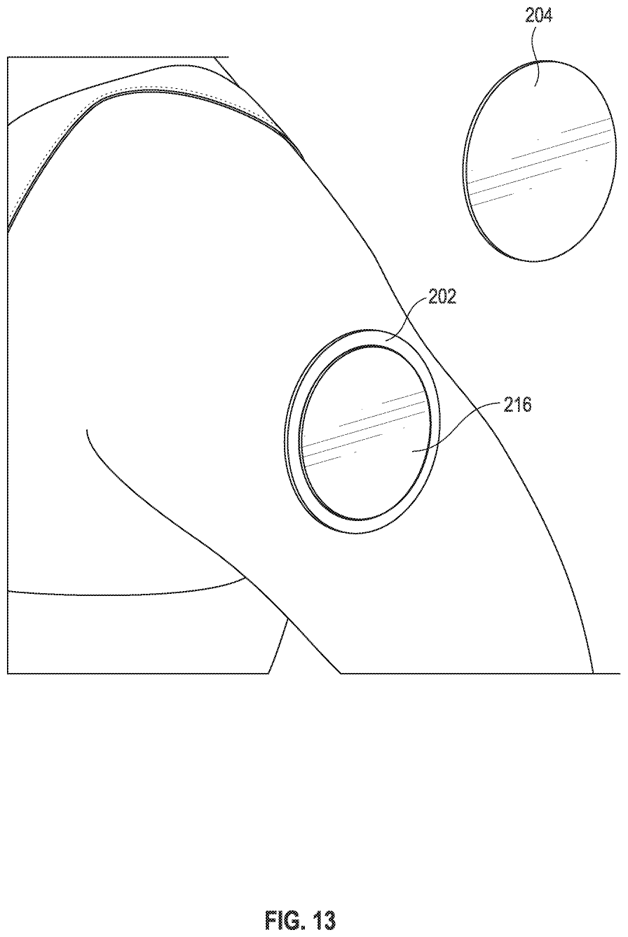

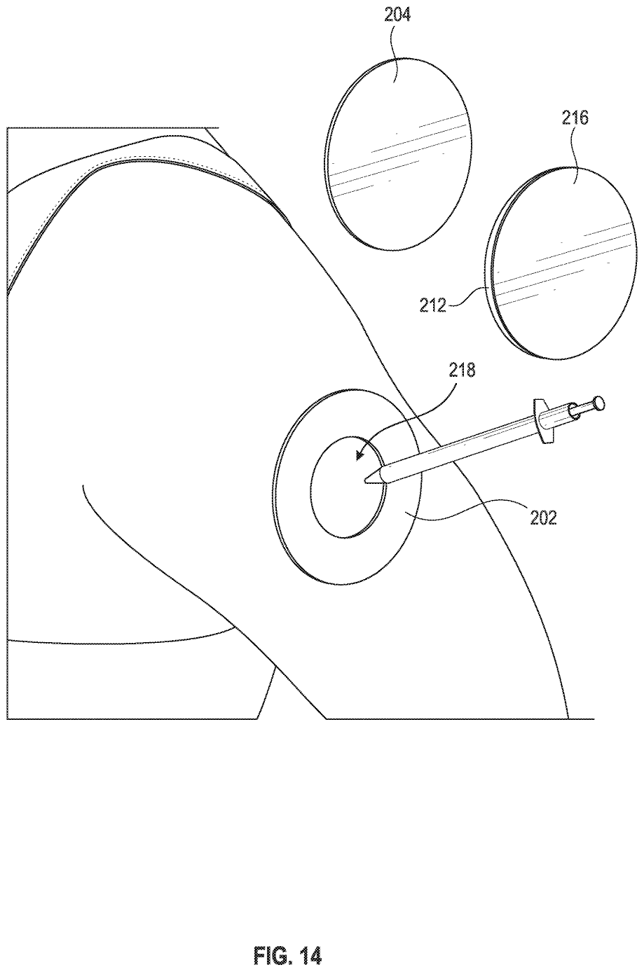

Accordingly, in some methods of use, a user will peel the release liner 204 from the first side 206 of the first member 202, exposing the adhesive on the first side 206. The user will then press the first side 206 onto the desired location on a patient, as shown in FIG. 13, adhering it to the patient. The medication 210 on the second member 208 then contacts and is absorbed into the skin. The medication 210 remains covered and protected by the third member 212 and the cover 216, preventing unwanted removal, contact, or contamination of the medication 210. Once an appropriate amount of time has elapsed for the medication 210 to have its desired effect (e.g., in the case of lidocaine, numbing the skin for an injection), a user may grasp and peel the third member 212 from the first member 202. Because the second member 208 is adhered to the third member 212, it likewise, is removed, along with the cover 216. As a result, the only remaining portion on the patient is the first member 202. A user may then cleanse the skin through the aperture 218 in preparation for the procedure. As shown in FIG. 14, the aperture 218 functions as a target or bullseye, allowing a user (e.g., physician) to easily administer an injection into the prepared area without guessing. While a medication 210 was discussed as being received on the second member 208, it will be appreciated that the second member may also be a transdermal patch with medication disposed therein.

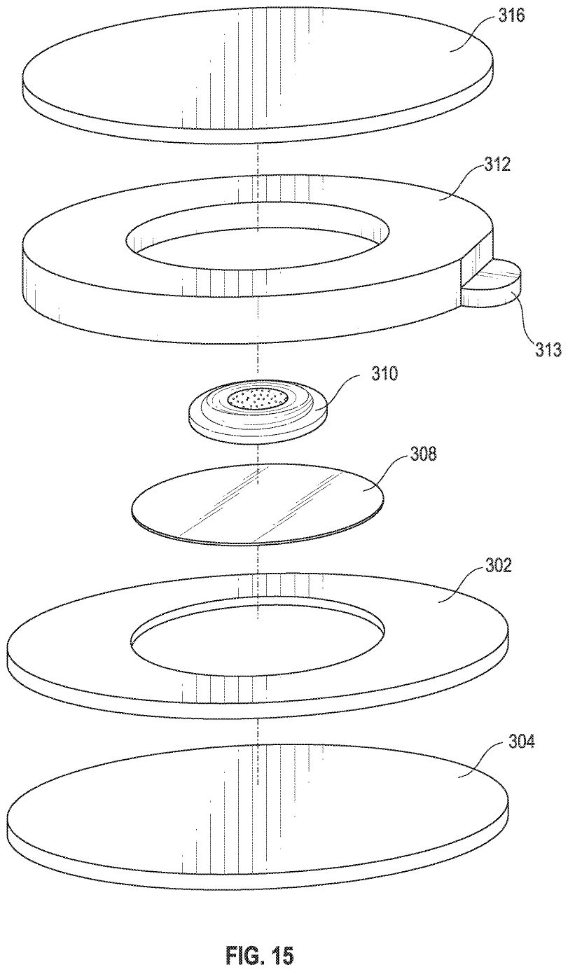

In some embodiments, as shown in FIG. 15, the third member 312 comprises a tab 313 that protrudes beyond the perimeter of the first member 302, allowing a user to easily grasp the tab 313 to peel the third member 312 from the first member 302. As in the prior embodiment, the adhesive bandage may comprise a release liner 304, a second member 308, a medication 310, and a cover 316.

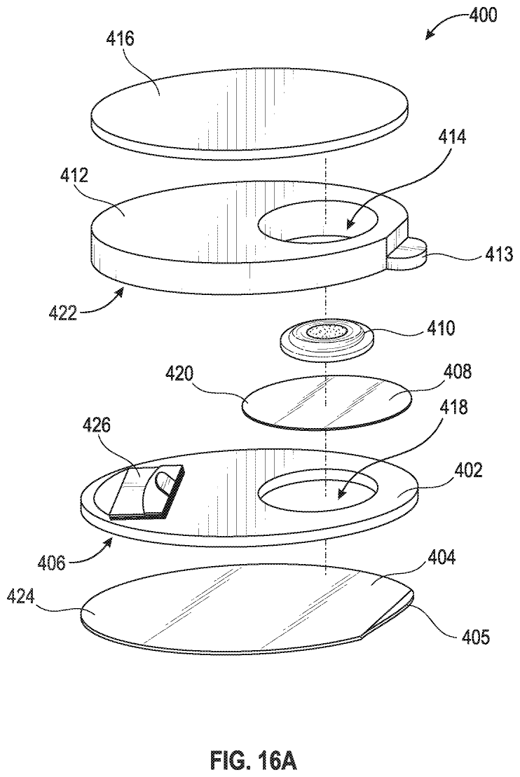

In some embodiments, as shown in FIGS. 16A-C, an adhesive bandage system 400 comprises a first member 402, a release liner 404 (e.g., backing paper) coupled to a first side 406 of the first member 402, and a second member 408 comprising medication or other topical ointment/cream 410. The second member 408 may comprise gauze or other non-stick material that allows for the medication to pass therethrough. A third member 412 may protect a pre-packaged medication 410, as well as while the medication 410 is in use on a patient. For example, the third member 412 may comprise an aperture 414 in the center thereof and have a height greater than the height of the medication 410 on the second member 408, thereby allowing the medication 410 to reside within the aperture 414 (a medication reservoir) during packaging and storage, reducing or preventing the medication 410 from coming into contact with a cover 416. The third member 412 may comprise foam or other materials, allowing it to flex, allowing for the adhesive bandage 400 to be used on different curves of a patient's body. The third member 412 is manufactured from non-absorbent materials so that the medication is not absorbed thereinto. Lastly, a film or cover 416 encloses and protects the medication 410 during storage and while in use.

The second member 408 is received within, or placed over, a first aperture 418 of the first member 402. Because the second member 408 is made from gauze, mesh, or other material allowing the medication 410 to pass or seep therethrough, the medication 410 will contact a patient through the aperture 418 once the release liner 404 is removed. The second member 408 is adhered (preferably non-releasably), around an outer edge 420, to the third member 412. The underside 422 of third member 412 is releasably adhered to top side 424 of the first member 402, allowing a user to remove the third member 412 by peeling it from the first member 402. The third member 412 may further comprise a tab 413 to allow a user to more easily peel it from the first member 402. Additionally, the release liner 404 may comprise a tab 405 to likewise allow for easier gripping and removal from the first side 406 of the first member 402. Further, a wound bandage 426 may be coupled to the first member 402 and configured to cover the aperture 418 once an injection has been administered.

In some embodiments, the wound bandage 426 may be folded, as shown in FIG. 16A so as to not interfere with the aperture 418 and medication 410 as it passes through the aperture 418. In other words, the wound bandage 426 is interposed between the first member 402 and the third member 412, remaining secured therebetween. After a user has peeled or otherwise removed the third member 412 from the first member 402, the bandage 412 is exposed and a first end remains adhered to the first member 402. A user may then unfold and extend the wound bandage 426, as shown in FIGS. 16B and 16C after the injection so as to protect the injection site. In some instances, additional creams, ointments, or other medications may be placed within the aperture 418 and onto the skin before the wound bandage 426 is extended to cover it. The wound bandage 426 may adhere on an opposite end to the first member 412, keeping it secured in place until a user removes the first member 402 from the skin. In other words, a release tab 428 may be removed from the second end 430 of the wound bandage 426, allowing it to be adhered to the first member 402. It will be appreciated that the port/couplers 114 shown in prior embodiments may also be used with this and any other embodiment disclosed herein.

While lidocaine was used as an example above, it will be appreciated that other medications/creams/ointments may be used. Indeed, in some instances, the diameter or other size of the apertures may vary to aid in dosing/uptake depending on the type of medication and the patient (e.g., age, weight, etc.). In some instances, the cover 216, 316, 416 is removably adherable, allowing a user to deposit the desired medication within the aperture 214, 314, 414 of the third member 212, 312, 412 and onto the second member 208, 308, 408.

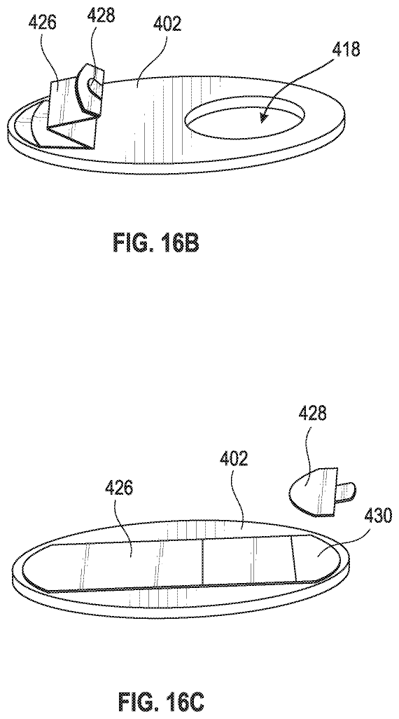

Referring to FIGS. 17-20, in some embodiments, an adhesive bandage system 500 comprises a first member 502 (e.g., medical tape) configured to adhere to skin of a patient, a removable center region 504, an aperture or treatment window 506 (exposed when the center region 504 is removed), a medication component 508, and a tactile distraction element 510 positioned around the treatment window 506. In some embodiments, the medication component 508 comprises a lidocaine patch or other topical anesthetic patch, and the lidocaine patch or other topical anesthetic patch itself forms the removable center region 504. In other words, the removable center region 504 and the medication component 508 need not be separate components. In other embodiments, the medication component 508 may comprise a topical anesthetic, a lidocaine patch, a medicated pad, a reservoir containing lidocaine or another numbing agent, or any other medication-delivery structure suitable for conditioning a treatment area before a needle stick or other procedure, and the removable center region 504 may carry the medication component 508, cover the medication component 508, or otherwise cooperate with the medication component 508 to maintain the medication component 508 in contact with the patient's skin before the procedure.

In some embodiments, the medication component 508 may be implemented as either a removable lidocaine patch or other topical anesthetic patch, or as a sealed medication reservoir (such as described in earlier embodiments) containing a topical medication, such as lidocaine cream, a lidocaine-containing formulation, gel, ointment, solution, or other topical anesthetic. When implemented as a sealed medication reservoir, the medication component 508 may be maintained in an airtight or substantially airtight storage state before use and may be opened, ruptured, unsealed, peeled, pressed, folded, or otherwise activated upon application to the skin 516. After activation, the removable center region 504, first member 502, cover, backing layer, release liner, reservoir wall, or other portion of the bandage system 500 may maintain the topical medication against the skin 516 in an occlusive or substantially occlusive delivery state until the removable center region 504 is removed to expose the treatment window 506.

In some embodiments of the bandage system 500, the sealed medication reservoir of the medication component 508 may be peel-activated or configured for peel-to-activate use. For example, a user may peel a release liner, cover, tab, removable center region 504, barrier layer, or other peelable layer to simultaneously break or open the airtight or substantially airtight storage seal and place or expose the topical medication for contact with the skin 516. The same peeling action may also expose adhesive for securing the first member 502 to the skin 516 or position the removable center region 504 relative to the treatment site. Accordingly, the bandage system 500 may be activated and applied in a single workflow step, rather than requiring separate actions to open a reservoir, dispense medication, and apply an occlusive cover.

In some embodiments, the removable center region 504 is removably coupled to the first member 502 and/or the tactile distraction element 510 by a perforated region 512, frangible connection, releasable adhesive, score, die-cut, kiss-cut, weakened portion, separable seam, or other removable coupling structure. The removable center region 504 may further comprise a pull tab 514 to facilitate grasping by a provider and removal of the removable center region 504 by breaking, peeling, tearing, lifting, or otherwise separating the removable center region 504 from the first member 502 and/or tactile distraction element 510, thereby exposing the treatment window 506 and the treated skin therebelow.

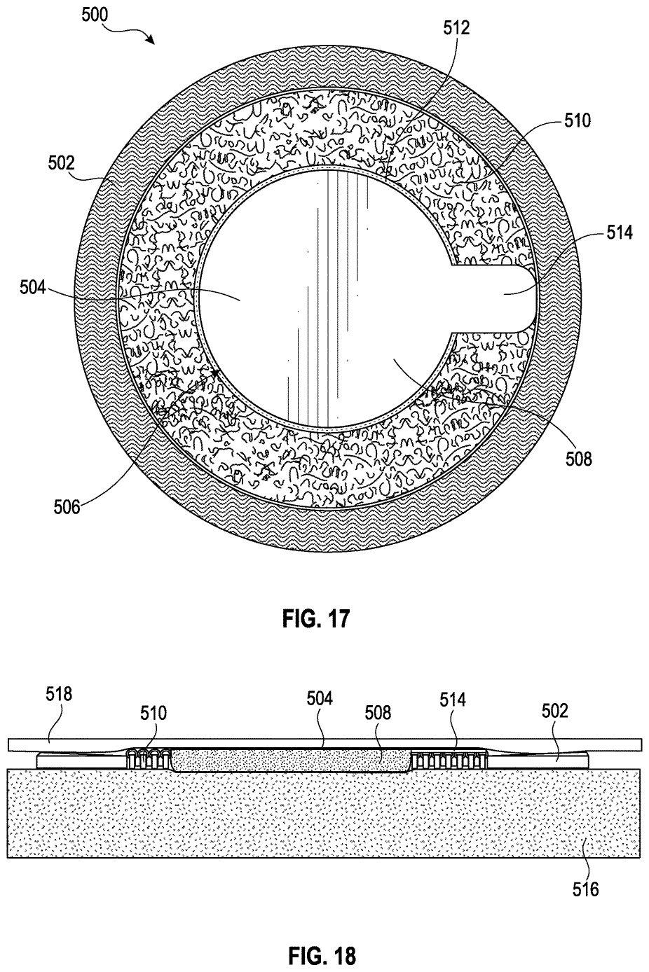

Referring to FIG. 18, in some embodiments, the adhesive bandage system 500 is shown adhered to a user's skin 516. This cross-sectional view shows the first member 502 adhered to the skin 516, thereby securing the bandage system 500 to the patient. The removable center region 504, which may comprise or be formed by the medication component 508, is in contact with the skin 516. For example, in some embodiments, the removable center region 504 is a lidocaine patch that contacts and conditions the skin 516. Likewise, the tactile distraction element 510 is in contact with the skin 516, but is not pressed into the skin 516. As shown in this embodiment, an outer bandage cover 518 (or release liner) may be configured to cover the entire adhesive bandage system 500. This helps prevent unwanted withdrawal of the removable center region 504 prior to removal by a provider and also ensures the medication component remains in a sterile environment. It will be understood that the underside may likewise comprise a release liner to thereby enclose the medication component 508, first member 502, and tactile distraction element 510 prior to use. This helps to ensure the adhesive on the first member 502 remains ready for use, and also maintains a sterile environment and to prevent unwanted contact of the medication component 508 prior to use.

Although FIGS. 17-24 schematically illustrate the medication component 508, it will be appreciated that the medication component 508 may include any of the sealed reservoir, release liner, cover, barrier layer, peelable layer, reservoir wall, seal, or occlusive delivery arrangements described with respect to earlier embodiments. Accordingly, the reservoir-based medication component 508 need not be illustrated in detail in FIGS. 17-24 to be included in the bandage system 500.

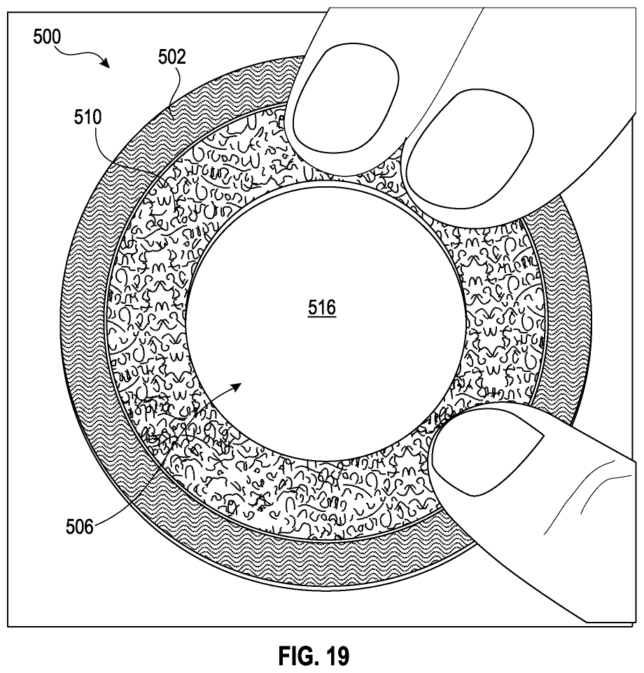

In use, the adhesive bandage system 500 may be applied to a patient so that the medication component 508 is positioned at an intended injection site on the skin 516. In embodiments where the medication component 508 forms the removable center region 504, the lidocaine patch or other medication patch may remain in place for a predetermined period of time to numb or otherwise condition the skin 516 and may then be removed as the removable center region 504. In other embodiments, the medication component 508 may remain in place with, under, or adjacent to the removable center region 504 for a predetermined period of time to numb or otherwise condition the skin 516. The removable center region 504 may then be removed from the first member 502 to expose the treatment window 506 and the conditioned skin 516, as best shown in FIG. 19. Because the first member 502 remains adhered to the skin 516 and the tactile distraction element 510 remains in contact with the skin 516 around the treatment window 506, the treatment window 506 clearly identifies the location that has been conditioned by the medication component 508 and provides a convenient access opening for cleansing, antiseptic application, visualization, and needle insertion.

The tactile distraction element 510 may be positioned in a ring, annulus, frame, partial ring, opposing side portions, or other surrounding arrangement around the treatment window 506. The surrounding geometry allows the provider to press on the tactile distraction element 510 while naturally stabilizing the patient's arm or other anatomical site. This pressure may activate the tactile distraction element 510 without requiring a separate device or a separate step. Accordingly, the tactile distraction element 510 may provide one-handed activation during ordinary provider workflow, while the treatment window 506 remains open for the injection.

In some embodiments, the tactile distraction element 510 comprises a plurality of projections, hooks, micro-hooks, bristles, raised textures, ridges, nodules, bumps, ribs, compressible structures, resilient protrusions, domes, snap domes, mechanical clickers, collapsible structures, or other tactile stimulation structures. In some embodiments, the tactile distraction element 510 may comprise a hook-and-loop material, such as a hook-side material, arranged around the treatment window 506. When pressed, the hook-like structures may contact or deflect against the patient's skin to create a plurality of non-painful tactile stimulation points adjacent to the injection site.

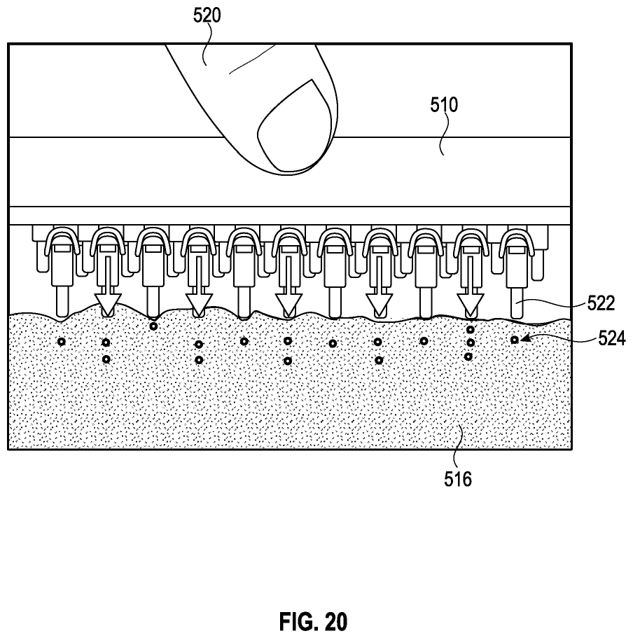

For example, as shown in FIG. 20, as the tactile distraction element 510 is pressed toward the skin 516 by a finger 520 of a provider, the stimulation structures 522 may contact, deflect against, compress, or otherwise stimulate the skin 516, thereby producing a nerve response 524. The nerve response 524 may provide competing sensory input adjacent to the treatment window 506 and may distract from a subsequent injection or other procedure performed through the treatment window 506.

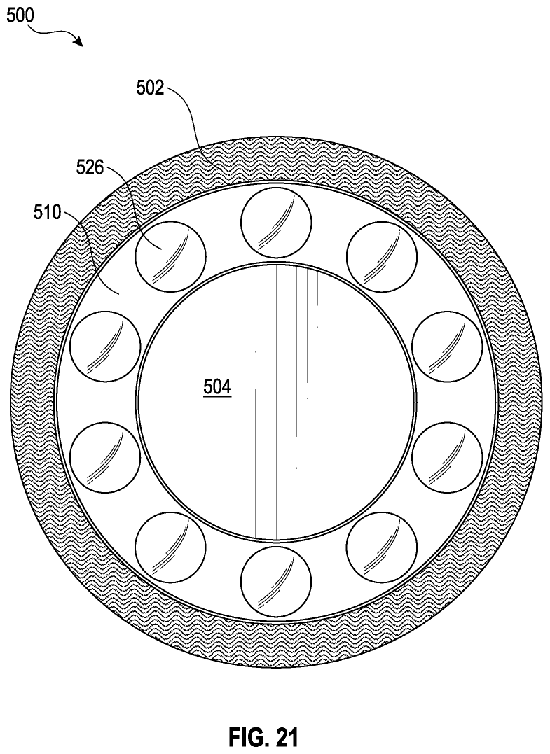

Referring to FIG. 21, in some embodiments, the tactile distraction element 510 may comprise one or more snap domes 526 or clicker structures that provide a tactile click, an audible click, a localized pressure pulse, or a combination thereof when depressed by the provider. The snap domes 526 may be distributed around the removable center region 504 and therefore the treatment window 506, arranged in a circular or polygonal pattern, positioned on opposing sides of the treatment window 506, or otherwise located so that provider pressure produces tactile stimulation near the injection site without obstructing the removable center region 504 and the treatment window 506. It will be appreciated that the snap domes 526 may be located on either or both sides of the bandage.

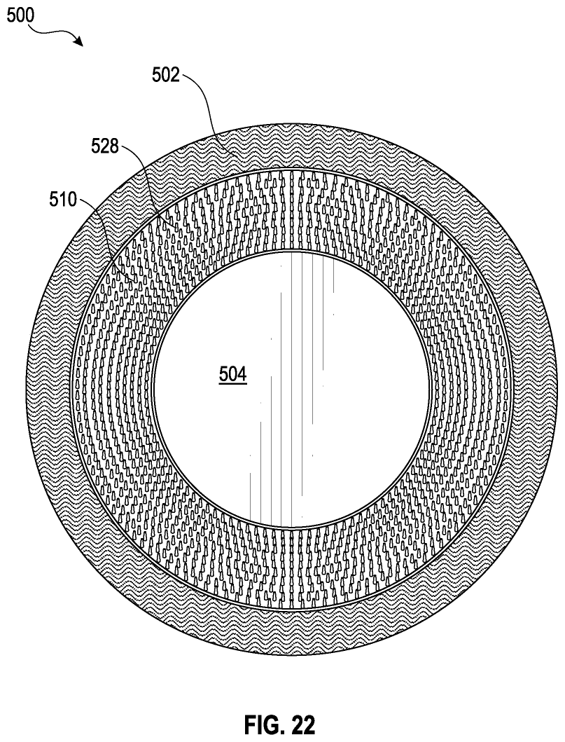

Referring to FIG. 22, in some embodiments, the tactile distraction element 510 may comprise micro-hooks 528 or other protrusions that provide a gentle prickly sensation. The micro-hooks 528 may be distributed around the removable center region 504 and therefore the treatment window 506, arranged in a circular or polygonal pattern, positioned on opposing sides of the treatment window 506, or otherwise located so that provider pressure produces tactile stimulation near the injection site without obstructing the removable center region 504 and the treatment window 506. As shown, it will be appreciated that the micro-hooks 528 may be located on a side of the bandage that contacts the skin, and that the opposite side (not visible in this view) may be smooth to facilitate a comfortable surface for a provider for applying pressure.

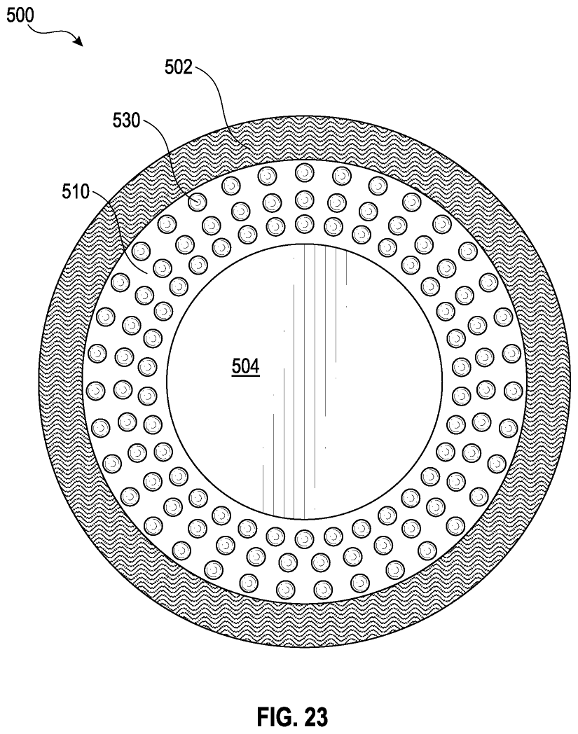

Referring to FIG. 23, in some embodiments, the tactile distraction element 510 may comprise microbeads 530 or other surface protrusions that provide a textured stimulation and sensation. The microbeads 530 may be distributed around the removable center region 504 and therefore the treatment window 506, arranged in a circular or polygonal pattern, positioned on opposing sides of the treatment window 506, or otherwise located so that provider pressure produces tactile stimulation near the injection site without obstructing the removable center region 504 and the treatment window 506. As shown, it will be appreciated that the microbeads 530 may be located on a side of the bandage that contacts the skin, and that the opposite side (not visible in this view) may be smooth to facilitate a comfortable surface for a provider for applying pressure.

In some embodiments, the tactile distraction element 510 may be formed from a polymeric material, elastomeric material, foam, nonwoven material, woven material, molded plastic, silicone, thermoplastic elastomer, adhesive-backed textured film, hook-and-loop material, or any other biocompatible material suitable for contacting or being positioned adjacent to the skin. The tactile distraction element 510 may be manufactured separately and coupled to the first member 502, or may be integrally formed with the first member 502 by molding, embossing, thermoforming, lamination, printing, coating, adhesive attachment, ultrasonic welding, heat sealing, or other manufacturing processes.

In some embodiments, the tactile distraction element 510 may be configured to provide tactile stimulation in response to a manual force applied by a provider, patient, caregiver, or other user. The tactile stimulation may include pressure, vibration, deflection, localized compression, resilient rebound, clicking, snapping, frictional stimulation, pricking sensation, textured contact, or combinations thereof. The tactile distraction element 510 may be configured to return toward an original position after pressure is released, may plastically deform, or may provide a single-use tactile response.

The adhesive bandage system 500 may provide combination therapy or sensory stacking by integrating a numbing component (medication component 508) and a tactile distraction element 510 into a single disposable bandage structure. The medication component 508 may reduce local sensitivity at the intended needle site, while the tactile distraction element 510 may provide competing tactile input adjacent to the needle site when the provider presses the surrounding structure. In other words, the provider presses on, or squeezes, the tactile distraction element 510 just prior to, or in combination with, the injection. The combined system may reduce perceived needle pain more effectively than either a numbing patch or a separate distraction device used alone, and may do so without batteries, reusable components, cleaning, storage, or separate positioning.

The removable center region 504 may provide additional workflow benefits. For example, removal of the removable center region 504, which may itself comprise the lidocaine patch or other medication component 508, may expose only the skin that has been conditioned by the medication component 508, thereby confirming the intended injection location. The treatment window 506 may allow a provider to cleanse the exposed skin with an antiseptic while maintaining the tactile distraction element 510 in a consistent position around the injection site. The treatment window 506 may also function as a visual target, bullseye, or site-preparation window so that the provider can inject into the prepared and numbed location rather than estimating the location after medication removal.

In some embodiments, the adhesive bandage system 500 further comprises a timing indicator configured to indicate that a predetermined amount of time has elapsed after the bandage system 500 is applied to the skin 516, after the medication component 508 contacts the skin 516, or after a medication reservoir is activated. The timing indicator may be positioned on the first member 502, removable center region 504, tactile distraction element 510, medication component 508, release liner, cover, tab, or another visible portion of the bandage system 500. The timing indicator may provide a visible color change, pattern change, opacity change, indicia appearance, indicia disappearance, gradient progression, or other visual output indicating that a desired treatment period, such as an anesthesia dwell period, has elapsed.

The timing indicator may comprise a moisture-activated indicator, pH-responsive indicator, thermochromic indicator, time-temperature indicator, body-temperature-activated indicator, soluble dye, migrating dye, wicking strip, printed ink, microencapsulated dye, silica gel particle, halochromic dye, thermochromic polymer ink, polydiacetylene material, hydrogel indicator, or combinations thereof. In some embodiments, the timing indicator is inactive during storage and begins changing only after exposure to skin moisture, skin pH, body temperature, pressure, oxygen, or another activation condition associated with application to the patient. The timing indicator may be calibrated to correspond to any suitable time period, including about 5 minutes, about 10 minutes, about 20 minutes, about 30 minutes, about 60 minutes, or a range between any of the foregoing, depending on the medication and procedure.

In some embodiments, the adhesive bandage system 500 further comprises a skin-contact confirmation indicator configured to indicate that the medication component 508 (lidocaine patch, medication reservoir, etc.), removable center region 504, first member 502, or another skin-facing portion is adequately contacting the skin 516. The skin-contact confirmation indicator may identify incomplete contact, air bubbles, lifting, poor adhesion, insufficient pressure, or areas where the medication is not in adequate contact with the skin. The skin-contact confirmation indicator may therefore assist a patient or provider in confirming that the topical anesthetic or other medication is properly positioned for delivery before the treatment period begins or while the treatment period is occurring.

In some embodiments, the timing indicator and the skin-contact confirmation indicator may be used with either a patch-based medication component or a reservoir-based medication component. For example, the indicators may be integrated with, printed on, coupled to, or positioned adjacent to a lidocaine patch, topical anesthetic patch, sealed medication reservoir, removable center region, release liner, cover, backing layer, or first member.

The skin-contact confirmation indicator may comprise a pressure-activated ink, pressure-rupturable microcapsules, carbonless-copy-type marking chemistry, contact dye, moisture-activated soluble dye, moisture-dissolvable stripe, pH-responsive material, adhesive-contact indicator, hydrogel indicator, transparent or translucent window, or combinations thereof. For example, a pressure-activated microcapsule ink may release or reveal a color when adequate pressure is applied between the bandage and the skin, while regions having air bubbles or poor adhesion may remain uncolored. In another example, a soluble dye stripe may dissolve, fade, or become transparent when it contacts moist skin. In another example, a transparent or translucent window may allow a provider to visually confirm skin blanching, medication spreading, skin contact, or another visible condition associated with proper placement or drug penetration.

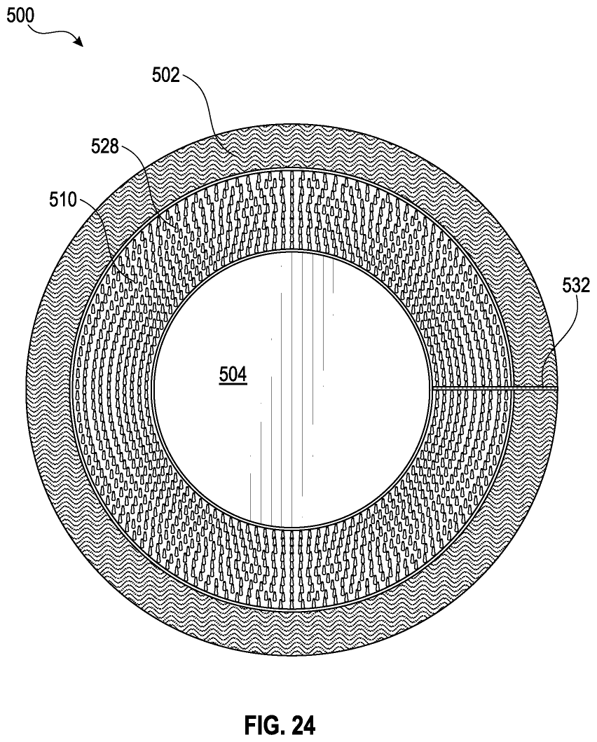

Referring to FIG. 24, in some embodiments, the adhesive bandage system 500 comprises a radial perforation 532, slit, separable seam, separable annulus, or other separation feature extending through at least a portion of the first member 502 and/or the tactile distraction element 510. The separation feature may allow the surrounding bandage structure to be opened or separated after placement of an intravenous line, catheter, tube, needle, or other medical implement through the treatment window 506. For example, the adhesive bandage system 500 may be placed on the skin so that the removable center region 504 overlies a desired treatment site. When a provider is ready to place an intravenous line or other medical implement, the provider may remove the removable center region 504 to expose the treatment window 506 and the underlying skin. After the exposed target area is cleansed or otherwise prepared, the intravenous needle, catheter, tube, or other medical implement may be inserted through the treatment window 506. Once the medical implement has been placed, the provider may remove the adhesive bandage system 500 by tearing, separating, opening, or otherwise disrupting the radial perforation 532 or other separation feature, thereby allowing the bandage structure to be removed from the patient without disturbing the placed intravenous line, catheter, tube, needle, or other medical implement. In some embodiments, the radial perforation 532 may be omitted and the surrounding bandage structure may instead include two ends that abut, overlap, interlock, releasably couple, or otherwise separate to permit removal around the medical implement.

In some embodiments, the adhesive bandage system 500 may be provided as a sterile, single-use medical article. One or more release liners may cover adhesive surfaces of the first member 502, the removable center region 504, the medication component 508, and/or the tactile distraction element 510 before use. The adhesive may comprise a medical-grade pressure-sensitive adhesive, hydrogel adhesive, silicone adhesive, acrylic adhesive, or other skin-compatible adhesive suitable for maintaining the bandage system 500 in position during medication delivery, skin preparation, tactile activation, and needle insertion.

Although certain embodiments are described in connection with lidocaine and injections, the medication component 508 may include other topical anesthetics, analgesics, antiseptics, antibiotics, vaccines, biologics, hormones, antihistamines, steroids, anti-inflammatory agents, or therapeutic agents, and the treatment window 506 may be used for injections, blood draws, intravenous access, vaccinations, dermatologic procedures, cosmetic procedures, biopsies, or other procedures in which pain reduction, site identification, and access to prepared skin are desirable. Further, the tactile distraction element 510 may be integrally formed with the first member 502, attached to the first member 502, laminated onto the first member 502, or printed, molded, embossed, adhered, or otherwise coupled to the first member 502. Additionally, the first member 502, timing indicator, skin-contact confirmation indicator, sealed reservoir, activation mechanism, and occlusive delivery structure may be used together or separately in any suitable combination. Additionally, while a perforated region 512 is shown and described for facilitating removal of the removable center region 504, it will be appreciated that any mechanism that allows a provider to remove it from the surrounding bandage portions (e.g., tactile distraction element 510 and first member 502) may be used without departing herefrom.

The bandage adhesive system 100 may be placed onto a finger, the abdomen, thigh, calf, arm, shoulder, or any other regions of exposed skin on the patient. Adhesives typically used in bandages and the like can be used herein.

As a result, the adhesive bandage system 100-500 disclosed herein solves the need for a bandage for use with creams, ointments, solutions, patches, topical anesthetics, and other medication-delivery structures that allow controlled dosing, maintain medication at a desired treatment site, identify the location of the applied medication, and assist with numbing, cleansing, targeting, tactile distraction, and access to a prepared treatment site before injections, intravenous access, or other medical procedures.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. An adhesive bandage system, comprising:
a first member comprising an adhesive surface configured to adhere to skin of a patient;
a removable center region removably coupled to the first member, the removable center region being removable to expose a treatment window through the first member;
a medication component positioned to condition a treatment site of the skin before removal of the removable center region, the medication component comprising a topical anesthetic patch or a medication reservoir containing a topical medication; and
a tactile distraction element coupled to the first member and positioned around at least a portion of the treatment window, the tactile distraction element configured to provide non-painful tactile stimulation adjacent to the treatment site in response to pressure applied to the tactile distraction element.

2. The adhesive bandage system of claim 1, wherein the medication component comprises the medication reservoir, and wherein the medication reservoir is peel-activated such that peeling a release liner, cover, tab, barrier layer, or other peelable layer opens or disrupts an airtight or substantially airtight storage seal and transitions the medication reservoir from an airtight or substantially airtight storage state to an occlusive or substantially occlusive delivery state in which the topical medication contacts the treatment site.

3. The adhesive bandage system of claim 2, wherein the medication reservoir is configured to reduce evaporation, drying, crystallization, oxidation, degradation, contamination, or moisture exchange before use.

4. The adhesive bandage system of claim 1, wherein the medication component comprises a lidocaine patch, topical anesthetic patch, lidocaine cream, lidocaine-containing formulation, cream, ointment, gel, or solution configured to numb the treatment site before a needle stick.

5. The adhesive bandage system of claim 1, wherein the tactile distraction element comprises a plurality of projections, hooks, micro-hooks, bristles, raised textures, ridges, nodules, bumps, ribs, compressible structures, resilient protrusions, domes, snap domes, mechanical clickers, collapsible structures, or combinations thereof.

6. The adhesive bandage system of claim 1, wherein the tactile distraction element comprises one or more snap domes configured to provide a tactile click, an audible click, a localized pressure pulse, or a combination thereof when depressed.

7. The adhesive bandage system of claim 1, wherein the tactile distraction element comprises micro-hooks or microbeads positioned on a skin-facing side of the tactile distraction element.

8. The adhesive bandage system of claim 1, wherein the removable center region is removably coupled to the first member or the tactile distraction element by a perforated region, frangible connection, releasable adhesive, score, die-cut, kiss-cut, weakened portion, or separable seam.

9. The adhesive bandage system of claim 1, wherein removal of the removable center region exposes only skin that was conditioned by the medication component and leaves the tactile distraction element adhered around the treatment window.

10. The adhesive bandage system of claim 1, further comprising a timing indicator configured to provide a visual indication that a predetermined treatment period has elapsed after application of the adhesive bandage system to the skin or, when the medication component comprises the medication reservoir, after activation of the medication reservoir.

11. The adhesive bandage system of claim 10, wherein the timing indicator comprises a moisture-activated indicator, pH-responsive indicator, thermochromic indicator, time-temperature indicator, soluble dye, migrating dye, printed ink, microencapsulated dye, silica gel particle, halochromic dye, thermochromic polymer ink, polydiacetylene material, or hydrogel indicator.

12. The adhesive bandage system of claim 1, further comprising a skin-contact confirmation indicator configured to visually indicate adequate contact between the medication component and the skin.

13. The adhesive bandage system of claim 12, wherein the skin-contact confirmation indicator comprises pressure-activated ink, pressure-rupturable microcapsules, contact dye, moisture-activated soluble dye, a moisture-dissolvable stripe, a pH-responsive material, an adhesive-contact indicator, a hydrogel indicator, a transparent window, or a translucent window.

14. A method of preparing and accessing a treatment site for a medical procedure using an adhesive bandage system, the method comprising:
adhering the adhesive bandage system to skin of a patient so that a medication component is positioned at an intended treatment site, the adhesive bandage system comprising a first member and a removable center region;
allowing the medication component to condition the intended treatment site;
removing the removable center region to expose a treatment window and the conditioned treatment site;
optionally applying pressure to a tactile distraction element positioned adjacent to the treatment window to provide non-painful tactile stimulation adjacent to the conditioned treatment site; and
performing the medical procedure through the treatment window.

15. The method of claim 14, wherein allowing the medication component to condition the intended treatment site comprises activating a medication reservoir to transition a topical medication from an airtight or substantially airtight storage state to an occlusive or substantially occlusive delivery state.

16. The method of claim 14, further comprising viewing a timing indicator to determine that a predetermined treatment period has elapsed before removing the removable center region.

17. The method of claim 14, further comprising viewing a skin-contact confirmation indicator to confirm adequate contact between the medication component and the skin.

18. The method of claim 14, further comprising cleansing or applying antiseptic to the conditioned treatment site through the treatment window after removing the removable center region and before performing the medical procedure.

19. The method of claim 14, wherein performing the medical procedure comprises inserting a needle, placing an intravenous line, drawing blood, administering a vaccination, performing a dermatologic procedure, performing a cosmetic procedure, or performing a biopsy through the treatment window.

20. An adhesive bandage system, comprising:
a first member configured to adhere to skin of a patient and defining a treatment window when a removable center region is removed;
the removable center region removably coupled to the first member and removable to expose the treatment window; and
a separation feature extending from an outer perimeter of the first member toward the treatment window, the separation feature configured to permit the adhesive bandage system to be opened or separated for removal from around a placed medical implement without disturbing the placed medical implement.
